(19) United States
(12) Patent Application Publication
(10) Pub. No.: US 2025/0288944 A1
(43) Pub. Date: Sep. 18, 2025

(54) THERMAL TREATMENT OF AN IONIC LIQUID MIXTURE OF A CONTAMINANT REMOVAL SYSTEM

(71) Applicant: Honeywell International Inc., Charlotte, NC (US)

(72) Inventors: Meghan Pipitone, Scottsdale, AZ (US); Zachery Clark, Sun City, AZ (US); Phoebe Henson, Glendale, AZ (US); David Loeffelholz, Long Beach, CA (US); Rebecca Kamire, Buffalo Grove, IL (US)

(21) Appl. No.: 18/752,474

(22) Filed: Jun. 24, 2024

Related U.S. Application Data
(60) Provisional application No. 63/566,027, filed on Mar. 15, 2024.

(51) Int. Cl. B01D 53/14; B01D 53/18; C07C 1/12

(52) U.S. Cl. CPC B01D 53/1425; B01D 53/1475; B01D 53/18; C07C 1/12; B01D 2252/30; B01D 2257/504

(57) ABSTRACT

A contaminant removal system includes a scrubber, an ionic liquid regeneration assembly, and a vacuum pump. The scrubber may be configured to absorb a contaminant from an air stream into an ionic liquid sorbent in an ionic liquid mixture. The ionic liquid regeneration assembly may be configured to desorb the contaminant from the ionic liquid sorbent under vacuum generated by the vacuum pump. The vacuum pump may be configured to transfer pump heat to the ionic liquid mixture.

### CROSS-REFERENCE TO RELATED APPLICATIONS

This application claims the benefit of U.S. Provisional Patent Application 63/566,027, filed Mar. 15, 2024, the entire contents of which are incorporated herein by reference.

### GOVERNMENT RIGHTS

This invention was made with Government support under Grant Contract Number 80MSFC18C0045 awarded by NASA Marshall Space Flight Center. The Government has certain rights in the invention.

### TECHNICAL FIELD

The present disclosure relates to systems and techniques for removing contaminants from an environment using contaminant removal systems.

### BACKGROUND

An environmental control system (ECS) may provide conditioned air to a passenger cabin (e.g., of a spacecraft) or other environment. Some of this conditioned air may be treated to remove contaminants. ECS components that are used to remove contaminants may be large and heavy, increasing an overall weight of the ECS, and may consume large amounts of power heating, cooling, and pressurizing various fluid streams.

### SUMMARY

The disclosure describes systems and techniques for removing contaminants using an ionic liquid sorbent of an ionic liquid mixture and transferring heat from a component (e.g., a vacuum pump) into the ionic liquid mixture to reduce a size and weight of an ECS. For example, because the ionic liquid mixture is heated by the absorbed heat and/or the component is cooled by transferring heat away from the component, an additional heater to heat the ionic liquid mixture or cooling mechanism to cool the component may not be required, or may be operated with lower energy consumption. A contaminant removal system may include a scrubber that removes contaminants from an air stream, such as a cabin air stream of a spacecraft, through absorption using an ionic liquid sorbent in an ionic liquid mixture, and an ionic liquid regeneration assembly that desorbs the contaminants from the ionic liquid sorbent. A vacuum pump drives desorption of the contaminants by generating vacuum in the ionic liquid regeneration assembly.

The vacuum pump generates pump heat in course of operation, for example, when generating the vacuum. In some examples, at least a portion of the ionic liquid mixture is transported through the vacuum pump, so that the ionic liquid mixture at least partially absorbs pump heat generated by the vacuum pump. In this way, a temperature of the vacuum pump may be maintained within operational specifications by transferring heat away from the vacuum pump. Further, because the ionic liquid mixture is at least partially heated by the pump heat, a dedicated heater to heat the ionic liquid mixture before regenerating the ionic liquid mixture may not be required, avoiding energy consumption associated with the heater and reducing system and operational complexity. In some examples, the system may include a heater as a supplementary heat source to further heat the ionic liquid mixture after being initially heated by absorbing heat from the vacuum pump. Such supplementary heating with a heater in combination with absorbing heat from the vacuum pump may consume less energy compared to solely heating the ionic liquid mixture with a heater.

As a result, the ionic liquid circuit may use less power to heat the ionic liquid mixture, and/or the vacuum pump used to drive desorption and process the contaminant may use less power and have a longer service life.

In some examples, the disclosure describes a contaminant removal system that includes a scrubber, an ionic liquid regeneration assembly, and a vacuum pump. The scrubber may be configured to absorb a contaminant from an air stream into an ionic liquid sorbent in an ionic liquid mixture. The ionic liquid regeneration assembly may be configured to desorb the contaminant from the ionic liquid sorbent under vacuum. The vacuum pump may be configured to generate the vacuum in the ionic liquid regeneration assembly. The vacuum pump may be further configured to transfer pump heat to the ionic liquid mixture.

In some examples, the disclosure describes a method for removing a contaminant from an environment that includes absorbing, by a scrubber, a contaminant from an air stream into an ionic liquid sorbent in an ionic liquid mixture. The method further may further include applying, by a vacuum pump, vacuum in an ionic liquid regeneration assembly. The method may further include desorbing, by an ionic liquid regeneration assembly, the contaminant from the ionic liquid sorbent under vacuum. The method may further include transferring pump heat from the vacuum pump to the ionic liquid mixture.

In some examples, the disclosure describes a contaminant removal system, comprising a scrubber, a stripper, and a vacuum pump. The scrubber may be configured to absorb a contaminant from an air stream into an ionic liquid sorbent in an ionic liquid mixture. The stripper may be configured to desorb the contaminant from the ionic liquid sorbent under vacuum. The vacuum pump may be configured to generate the vacuum in the ionic liquid regeneration assembly. The vacuum pump defines a liquid inlet, a liquid outlet, and a liquid passage between the liquid inlet and the liquid outlet. The scrubber may be fluidically coupled to the liquid inlet of the vacuum pump. The stripper may be fluidically coupled to the liquid outlet of the vacuum pump. The vacuum pump may be configured to transfer pump heat to the ionic liquid mixture along the liquid passage. The liquid passage is isolated from vacuum of the vacuum pump.

### BRIEF DESCRIPTION OF THE FIGURES

The details of one or more examples are set forth in the accompanying drawings and the description below. Other features, objects, and advantages will be apparent from the description and drawings, and from the claims.

FIG. 1 is a schematic diagram illustrating an example vacuum pump configured to transfer heat to an ionic liquid mixture.

FIG. 2 is a block diagram illustrating an example contaminant removal system including a scrubber, an ionic liquid regeneration system, and a vacuum pump for removing contaminants using an ionic liquid mixture.

FIG. 3 is a flow diagram illustrating an example method for removing a contaminant from an environment.

FIG. 4 is a schematic diagram illustrating an example contaminant removal system that includes a vacuum pump between a scrubber and a stripper.

### DETAILED DESCRIPTION

A contaminant removal system includes a scrubber that removes contaminants from an air stream, such as a cabin air of a spacecraft, through absorption using an ionic liquid sorbent in an ionic liquid mixture and an ionic liquid regeneration assembly (e.g., including a stripper) that desorbs the contaminants from the ionic liquid sorbent. A vacuum pump used to apply vacuum in the ionic liquid regeneration assembly generates pump heat in the course of operation (e.g., while generating the vacuum to drive desorption by the stripper). At least a portion of the ionic liquid mixture is caused to flow through the vacuum pump to at least partially absorb the pump heat. In this way, the vacuum pump may be cooled, and the ionic liquid mixture may be heated. Additional thermal management may not be required, or may be operated with lower energy consumption, to cool the vacuum pump or heat the ionic liquid mixture, reducing the complexity of the system and energy utilization, compared to a system in which pump heat is not absorbed by the ionic liquid mixture. As a result, the contaminant removal system may use less power to heat the ionic liquid mixture, and/or the vacuum pump used to desorb and process the contaminants from the stripper may use less power and have a longer service life.

Contaminant removal systems described herein may be utilized as part of an environmental control system (ECS), such as in spacecraft, aircraft, watercraft, and the like. In some examples, contaminant removal systems may be used in an ECS of a resource-limited environment, such as a passenger cabin of a spacecraft, in which carbon dioxide and water may be recycled to produce oxygen gas, water, methane, and a variety of other compounds used in life support systems. Such resource-limited environments may be particularly suited for a contaminant removal system that includes components that use low amounts of power and have extended service lives to reduce overall weight, power consumption, and maintenance load.

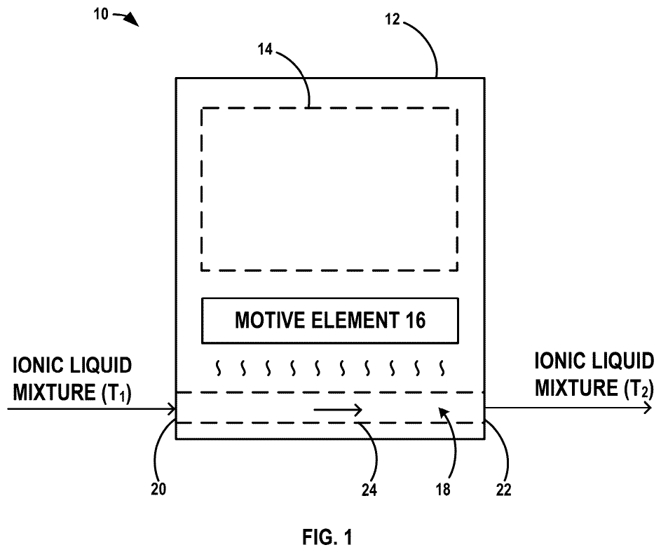

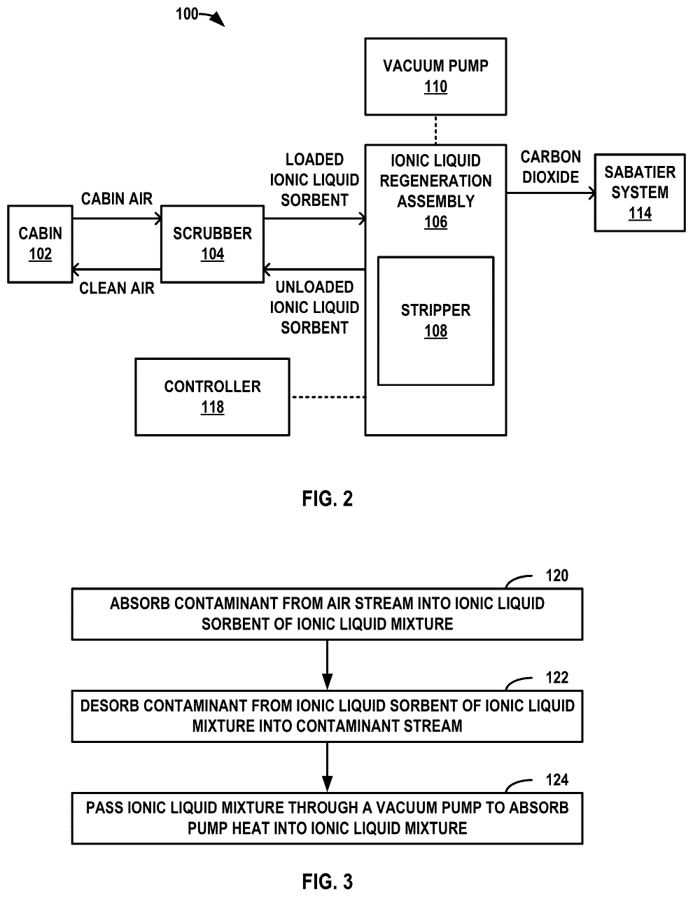

FIG. 1 is a schematic diagram illustrating an example vacuum pump 10 configured to transfer heat to an ionic liquid mixture. Vacuum pump 10 may be used to generate vacuum for a contaminant removal system, for example, in an ionic liquid regeneration assembly (e.g., as described with reference to FIGS. 2 and 3). For example, vacuum pump 10 includes a housing 12 defining a vacuum chamber 14 and a motive element 16. Motive element 16 may generate vacuum in vacuum chamber 14, and vacuum chamber 14 may be coupled to the ionic liquid regeneration assembly to draw vacuum from the ionic liquid regeneration assembly. For example, motive element 16 may include a motor, a rotor, a stator, a compressor, or any suitable element configured to generate vacuum.

In course of operation, vacuum pump 10 may generate pump heat, for example, when generating the vacuum. For example, motive element 16 may generate heat by friction or flow phenomena. If not transferred away from vacuum pump 10, such pump heat may cause the temperature of vacuum pump 10 to rise, which may affect the operation and service life of vacuum pump 10.

In some examples, vacuum pump 10 is configured to transfer pump heat to the ionic liquid mixture, and the ionic liquid mixture may be used to absorb the pump heat, and ultimately transport the pump heat away from vacuum pump 10. For example, vacuum pump 10 may define a liquid passage 18, and the ionic liquid mixture may flow through the liquid passage 18. In some examples, vacuum pump 10 defines a liquid inlet 20, a liquid outlet 22, with liquid passage 18 between the liquid inlet 20 and the liquid outlet 22. Pump heat may be absorbed by the ionic liquid mixture as the ionic liquid mixture flows along liquid passage 18, and thus, vacuum pump 10 may be configured to transfer the pump heat to the ionic liquid mixture along the liquid passage.

Liquid passage 18 is isolated from vacuum of vacuum pump 10. For example, liquid passage 18 may be fluidically isolated from vacuum chamber 14, or otherwise from any vacuum line or stream. Nonetheless, liquid passage 18 may be thermally coupled to vacuum chamber 14 or to other portions of vacuum pump 10 under vacuum.

In some examples, vacuum pump 10 includes a liquid conduit 24 defining liquid passage 18. Liquid conduit 24 may be coupled to, or may be define, liquid inlet 20 and/or liquid outlet 22. Liquid conduit 24 may include at least one metal or alloy, or any composition that allows heat transfer to the ionic liquid mixture within liquid passage 18. Liquid conduit 24 may include conduit walls of any suitable cross-section, for example, circular, elliptical, curved, polygonal, or combinations thereof. In some examples, liquid conduit 24 includes one or more tubes. The cross-sectional shape or size may be substantially constant along liquid conduit 24, or may vary along liquid conduit 24. For example, at least a portion of liquid conduit 24 may include a converging portion, a diverging portion, or a varying diameter.

Liquid conduit 24 may extend along any suitable path through vacuum pump 10, for example, along a path that includes a straight portion, a curved portion, an undulating portion, a zig-zag portion, a U-portion, or any combinations thereof. In some examples, liquid conduit 24 includes a single straight tube. In other examples, liquid conduit 24 includes a plurality of tubes. In some examples, liquid conduit 24 is adjacent motive element 16, or may surround a portion of motive element 16.

While liquid passage 18 may be within vacuum pump 10 (for example, within housing 12 of vacuum pump 10), in other examples, at least a portion of liquid passage 18 may be external to vacuum pump 10. For example, at least a portion of liquid conduit 24 (or another conduit) may be positioned along an exterior surface of housing 12, and the ionic liquid may flow through liquid passage 18 within liquid conduit 24 (or another conduit) along the exterior surface.

Thus, an ionic liquid mixture passing through liquid passage 18 may absorb pump heat from vacuum pump 10. For example, the ionic liquid mixture may be introduced into liquid inlet 20 at a first temperature (T₁) and may be discharged from liquid outlet 22 at a second temperature (T₂) greater than the first temperature. The difference between T₁ and T₂ may depend on a number of geometric and operational parameters, for example, an interior volume of liquid passage 18, a flow rate of the ionic liquid, a path length of conduit 24, power or wattage of vacuum pump 10, or the extent of vacuum generated by motive element 16 in vacuum chamber 14, which in turn may affect the extent of heat generated by motive element 16.

Vacuum pumps configured to transfer pump heat to an ionic liquid mixture may be used in contaminant removal systems, as described with reference to FIG. 2.

FIG. 2 is a block diagram illustrating an example contaminant removal system 100 including a scrubber 104, an ionic liquid regeneration system 106, and a vacuum pump 110 for removing contaminants from an environment. Contaminant removal system 100 is configured to remove contaminants from a cabin 102. Cabin 102 may be a controlled environment, such as an aircraft cabin, spacecraft cabin, watercraft cabin, or the like, and contaminants removed from cabin 102 may include, but are not limited to, carbon dioxide, water, hydrocarbons, permanent gases, or the like. In the example of FIG. 2, cabin 102 is a cabin of a closed-loop system, such as a spacecraft cabin or submarine cabin, in which components of a cabin air stream from cabin 102, such as carbon dioxide and water, may be removed within contaminant removal system 100, allowing a purified supply air stream to be generated and carbon dioxide and water to be recovered. However, in other examples, cabin 102 may be a cabin of an open-loop system, such as an aircraft cabin, in which components of a cabin air stream may be removed to generate a purified supply air stream with only partial or no subsequent recovery of the contaminants.

In examples in which the contaminant includes carbon dioxide, contaminant removal system 100 may include a Sabatier system 114 configured to convert the carbon dioxide to methane. Sabatier system 114 may be configured to react the contaminant with hydrogen to produce water and methane and either dry the methane for storage as fuel or send the methane to other reactors for further conversion. For example, while not shown in FIG. 2, system 100 may include a methane pyrolysis system configured to convert methane to carbon and hydrogen, and subsequently return at least a portion of the hydrogen to Sabatier system 114 to produce more methane and water. Sabatier system 114 may be configured to send the water to an electrolysis system to produce hydrogen and oxygen, contributing to a more closed loop O₂/CO₂ cycle. In other examples, Sabatier system 114 may further pressurize the methane to above ambient pressure to form methane for rocket fuel.

System 100 is configured to remove the contaminants using an ionic liquid sorbent. Ionic liquid sorbents may be salts that are generally comprised of an anion and an organic cation. These salts may be liquid at their temperature of use, have effectively zero vapor pressure, be generally nontoxic, and/or have sufficient stability to resist deterioration. In some examples, liquid sorbents may contain relatively large organic cations and any of a variety of anions, which may be tailored to obtain desired characteristics, such as characteristics that improve absorption of the contaminant under operating conditions of system 100 and/or exclusion of the ionic liquid sorbent by a filtration membrane. Ionic liquid sorbents may be water soluble, hygroscopic (i.e., capable of absorbing moisture from the air), and/or capable of releasing water by evaporation, such as by elevating the temperature or reducing the water partial pressure. In system 100, the ionic liquid sorbent is dissolved in water to form an ionic liquid mixture. A mass fraction (or concentration) of ionic liquid sorbent in the ionic liquid mixture may be sufficiently high to remove contaminants and sufficiently low that the ionic liquid sorbent remains in solution through operating ranges (e.g., temperature range, pH range) and/or maintains a low viscosity.

Contaminant removal system 100 includes scrubber 104. Scrubber 104 is configured to absorb one or more contaminants from a cabin air stream into the ionic liquid sorbent and discharge a clean air stream to cabin 102. Scrubber 104 is configured to receive unloaded ionic liquid sorbent having a relatively low concentration of contaminants, contact the unloaded ionic liquid sorbent with cabin air through one or more separation membranes to absorb the contaminants into the ionic liquid sorbent, and discharge loaded ionic liquid sorbent that includes a higher concentration of contaminants.

Contaminant removal system 100 includes an ionic liquid regeneration assembly 106. Ionic liquid regeneration assembly 106 includes a stripper 108 configured to desorb one or more contaminants from the loaded ionic liquid sorbent and discharge the contaminants from stripper 108, such as to a Sabatier system 114. Stripper 108 is configured to receive the loaded ionic liquid sorbent from scrubber 104, contact the loaded ionic liquid sorbent with a sweep gas stream or other low pressure volume through one or more separation membranes to desorb the contaminant from the ionic liquid sorbent, and discharge unloaded liquid sorbent that includes a lower concentration of the contaminant. To desorb the contaminant from the ionic liquid sorbent, the gas phase side of stripper 108 may be at a vacuum. Ionic liquid regeneration assembly 106 includes a vacuum pump 110 configured to generate the vacuum to desorb the contaminant from the ionic liquid sorbent and pressurize the contaminant, such as for further storage or processing. Vacuum pump 110 may be the same as or similar to vacuum pump 10 described with reference to FIG. 1.

As described with reference to FIG. 1, vacuum pump 110 may generate pump heat. During operation of system 100, the pump heat may be absorbed into the ionic liquid mixture, for example, by passing ionic liquid mixture through vacuum pump 110, thereby transporting heat away from vacuum pump 110. Thus, the temperature of the ionic liquid mixture may increase by absorbing the pump heat from vacuum pump 110. Such an increase in the temperature of the ionic liquid mixture may facilitate absorption of the contaminant by the ionic liquid mixture. Further, transporting pump heat away from vacuum pump 110 may promote maintaining vacuum pump 110 within a predetermined operating temperature range, which may reduce maintenance costs and increase a service life of vacuum pump 110.

Contaminant removal system 100 may include a process control system that includes a controller 118 and one or more sensor sets (not shown). Controller 118 may be configured to receive measurements from the one or more sensor sets and/or components of contaminant removal system 100 and/or send control signals to components of contaminant removal system 100. Controller 118 may be communicatively coupled to and configured to receive measurement signals from the one or more sensor sets, and other process control components (not shown) of contaminant removal system 100, such as: control valves for various streams; pumps; heaters; heat exchangers; compressors; and the like. The sensor sets may include instrumentation configured to detect any of a pressure, temperature, flow rate, and/or contaminant concentration (e.g., carbon dioxide concentration or water concentration) of a liquid or gas stream of contaminant removal system 100. Controller 118 may be configured to use the detected conditions to control operation of contaminant removal system 100 to function as described in the application.

Controller 118 may include any of a wide range of devices, including control circuitry, processors (e.g., one or more microprocessors, one or more application specific integrated circuits (ASICs), one or more field programmable gate arrays (FPGAs), or the like), processing circuitry, one or more servers, one or more desktop computers, one or more notebook (i.e., laptop) computers, one or more cloud computing clusters, or the like.

In some examples, controller 118 is configured to control a contaminant concentration within the environment of cabin 102. For example, controller 118 may be configured to receive a contaminant concentration measurement for a contaminant, such as from cabin air sensor set or a contaminant concentration sensor in cabin 102. Controller 118 may be configured to determine whether the contaminant concentration measurement exceeds a contaminant concentration setpoint. For example, the contaminant concentration setpoint may be a target concentration of the supply air stream for maintaining cabin 102 below a threshold contaminant concentration. Controller 118 may be configured to send, in response to the contaminant concentration measurement exceeding the contaminant concentration setpoint, a control signal to decrease a concentration of the contaminant in the supply air stream. For example, controller 118 may send a control signal to control a flow rate of the ionic liquid mixture; a flow rate, humidity, and/or temperature of a sweep gas stream into stripper 108; a temperature of the ionic liquid mixture at scrubber 104 or stripper 108; a flow rate of the cabin air stream; or any other variable that may control a rate of removal of contaminants from the cabin air stream.

FIG. 3 is a flow diagram illustrating an example method for removing a contaminant from an environment, described with respect to system 100 of FIG. 2. However, the method of FIG. 3 may be performed using any suitable system. To remove contaminants from cabin 102, the method includes absorbing, by scrubber 104, a contaminant from an air stream into an ionic liquid sorbent in an ionic liquid mixture (120). For example, controller 118 may detect that the cabin air stream has a concentration of one or more contaminants that is above a threshold and, in response, control components of a cabin air circuit to control a flow rate of the cabin air stream and components of an ionic liquid circuit to control a flow rate and temperature of the ionic liquid mixture. As a result, a clean air stream has a lower concentration of the contaminant than the cabin air stream. For example, the clean air stream may have a concentration of a contaminant that is about 25% to about 99% less than a concentration of the contaminant in the cabin air stream.

To recover the contaminant from the ionic liquid sorbent, the method includes desorbing, by ionic liquid regeneration assembly 106, the contaminant from the ionic liquid sorbent (122). For example, controller 118 may control components of an ionic liquid circuit to control a flow rate of ionic liquid sorbent between scrubber 104 and stripper 108 and control components for processing a contaminant stream, such as vacuum pump 110, to create a pressure differential across stripper 108 to cause stripper 108 to desorb the contaminant from the ionic liquid sorbent.

To cool vacuum pump 110 and/or heat the ionic liquid mixture, the method may further include passing the ionic liquid mixture through vacuum pump 110 to absorb pump heat from vacuum pump 110 (124). Controller 118 may control one or more components of an ionic liquid circuit to control various parameters, such as a flow rate of ionic liquid mixture through vacuum pump 110, temperature of the ionic liquid mixture, to control rate of heat transfer from vacuum pump 110 to the ionic liquid mixture. For example, controller 118 may control a pump to control the flow rate of the ionic liquid mixture. Passing the ionic liquid mixture (124) may include introducing the ionic liquid mixture from the scrubber 104 at a first temperature into vacuum pump 110, and allowing the ionic liquid mixture to heat to a second temperature greater than the first temperature by absorbing the pump heat. The method may further include receiving, by stripper 108, the ionic liquid mixture at a temperature greater than first temperature from vacuum pump 110. For example, stripper 108 may receive the ionic liquid mixture at the second temperature, or at a temperature lower than the second temperature but greater than the first temperature. In some examples, the technique may further include heating the ionic liquid mixture to a temperature greater than the second temperature, for example, with heater 230 downstream of scrubber 104 and upstream of stripper 108. Thus, stripper 108 may receive the ionic liquid mixture at a temperature greater than the second temperature by supplemental heating of the ionic liquid mixture after the ionic liquid mixture is heated by vacuum pump 110 (124).

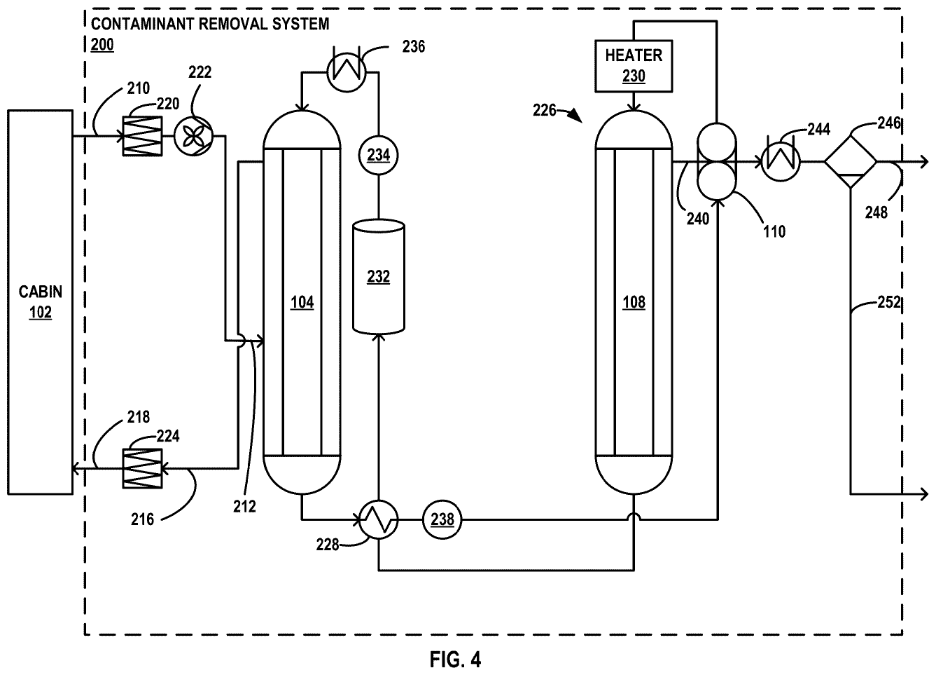

FIG. 4 is a schematic diagram illustrating an example contaminant removal system 200 that includes vacuum pump 110 between scrubber 104 and stripper 108. Contaminant removal system 200 includes a cabin air circuit configured to circulate cabin air between cabin 102 and scrubber 104. In the example of FIG. 4, cabin air stream 210 includes a filter 220 configured to remove particulates from cabin air stream 210 prior to entry into scrubber 104 and a blower 222 configured to draw cabin air into scrubber 104, while a supply air stream 218 includes a filter 224 configured to remove any leaked ionic liquid sorbent and/or further filter clean air from supply air stream 218 prior to entry into cabin 102.

Contaminant removal system 200 includes an ionic liquid circuit 226 configured to circulate ionic liquid sorbent between scrubber 104 and stripper 108. For example, a pump 234 may pump unloaded ionic liquid sorbent from an ionic liquid storage 232 and/or stripper 108 into scrubber 104. Unloaded ionic liquid sorbent may include unused ionic liquid sorbent free of contaminants or regenerated ionic liquid sorbent having a lower concentration of contaminants than the loaded ionic liquid sorbent. In some examples, the unloaded ionic liquid sorbent may be cooled by a cooler 236 prior to entry into scrubber 104. In some examples, the loaded ionic liquid sorbent may be pumped by a pump 238 and/or preheated by a heat exchanger 228 and/or heater 230 prior to entry into stripper 108.

Scrubber 104 is configured to absorb the contaminant from cabin air stream 212 into an ionic liquid sorbent and discharge a clean air stream 216 to cabin 102. On a gas phase side, scrubber 104 is configured to receive cabin air from cabin air stream 212 that includes contaminant species from cabin 102, such as carbon dioxide, water, hydrocarbon volatiles, permanent gases, and other gaseous substances. Scrubber 104 is configured to absorb one or more contaminant species in the cabin air into an ionic liquid sorbent. Scrubber 104 includes one or more separation membranes, each configured to flow (e.g., provide or direct flow of) cabin air from cabin air stream 212 on a gas phase side (e.g., a tube side) of the respective membrane and flow an ionic liquid sorbent on a liquid phase side (e.g., a shell side) of the membrane. Contaminants may pass through the membrane due to a concentration gradient between the cabin air and the ionic liquid sorbent and become absorbed by the ionic liquid sorbent, while the ionic liquid sorbent may not substantially flow through the membrane. As a result, clean air from clean air stream 216 discharged from scrubber 104 may have a lower concentration of contaminants than cabin air from cabin air stream 212 received by scrubber 104. Scrubber 104 is configured to discharge a clean air stream 216 to cabin 102.

On a liquid phase side, scrubber 104 is configured to receive unloaded ionic liquid sorbent, such as from ionic liquid storage 232. The unloaded ionic liquid sorbent may flow through scrubber 104 and absorb contaminants from cabin air through the membrane(s) of scrubber 104. As a result, the loaded ionic liquid sorbent discharged from scrubber 104 may have a higher concentration of contaminants than the unloaded ionic liquid sorbent received by scrubber 104. Scrubber 104 may discharge the loaded ionic liquid sorbent containing the contaminants to stripper 108.

Contaminant removal system 100 includes stripper 108 downstream of scrubber 104. Stripper 108 is configured to desorb the contaminant from the ionic liquid sorbent into a contaminant stream 240. On a liquid phase side, stripper 108 is configured to receive loaded ionic liquid sorbent from scrubber 104 and desorb one or more contaminants from the loaded ionic liquid sorbent. Stripper 108 includes one or more membranes, each configured to flow the loaded ionic liquid sorbent on one side (e.g., a shell side) of the membrane and contaminated air to a contaminant stream 240 on an opposite side (e.g., a tube side) of the membrane. Contaminants may flow across fibers of the membrane due to a concentration gradient, while the ionic liquid sorbent may not substantially flow across the fibers of the membrane. As a result, unloaded ionic liquid sorbent discharged from stripper 108 may have a lower concentration of contaminants than the loaded ionic liquid sorbent received by stripper 108. On a gas phase side, stripper 108 is configured to discharge the contaminant in a contaminant stream 240. Contaminant stream 240 may be continuously removed from stripper 108 to assist migration of the contaminants from the loaded ionic liquid sorbent into contaminant stream 240.

Scrubber 104 and/or stripper 108 may include one or more membrane separators configured to flow air on a first side and flow ionic liquid sorbent on a second, opposite side. For example, a membrane separator may include a plurality of parallel membrane contactors. In some examples, a membrane contactor may include a cylindrical module filled with parallel or woven hollow porous fibers. For example, dimensions of these hollow fibers could be less than about 3 mm, and the pore dimension could be less than about 2 microns. The high surface area of the hollow fiber membrane contactors enables a high mass transfer of contaminant gasses, such as carbon dioxide and water, into the ionic liquid sorbent using a relatively small system volume and weight. The material of the hollow fibers can be selected such that the ionic liquid sorbent does not wet the pores, and the trans-membrane pressure is kept sufficiently low to prevent pore penetration. As a result, the membrane contactor may ensure that the ionic liquid sorbent and gas stream do not need further separation, such that contaminant removal system 200 may act in a gravity-independent way without the use of moving parts. Fiber materials may include, but are not limited to, hydrophobic materials such as polypropylene, polyvinylidene fluoride, polysulfone, polyimide, polytetrafluoroethylene (PTFE), and the like. In some examples, a coating may be applied to reduce liquid flow through the pores. Coatings that may be used include, but are not limited to, PTFE, a crosslinked siloxane, perfluorinated polymers, functionalized nanoparticles, and the like to prevent liquid flow through the pores. While described in FIG. 4 as flowing through a “tube” side, ionic liquid sorbent flow can be either on the “tube” side or the “shell” side, while air is flowed on the opposite side.

Vacuum pump 110 is configured to generate the vacuum in stripper 108 to drive or facilitate desorption of contaminant. Vacuum pump 110 is positioned downstream of scrubber 104 and upstream of stripper 108. A liquid inlet of vacuum pump 110 may be fluidically coupled to scrubber 104 to receive the ionic liquid mixture from scrubber 104 at a first temperature, and a liquid outlet of vacuum pump 110 may be configured to discharge the ionic liquid mixture at a second temperature greater than the first temperature. Stripper 108 may be fluidically coupled to the liquid outlet of vacuum pump 110 to receive the ionic liquid mixture at a temperature greater than the first temperature. For example, stripper 108 may directly receive the ionic liquid mixture from the liquid outlet of vacuum pump 110 at the second temperature, or at a temperature lower than the second temperature but greater than the first temperature. In some examples, stripper 108 may receive the ionic liquid mixture at a temperature greater than the second temperature, for example, being heated by a heater between the liquid outlet of vacuum pump 110 and stripper 108.

Because vacuum pump 110 at least partially heats the ionic liquid mixture flowing from scrubber 104 to stripper 108, the power consumed to heat the ionic liquid mixture and/or cool vacuum pump 110 is reduced. In some examples, system 200 does not include heater 230 or any heater configured to heat the ionic liquid mixture upstream of stripper 108 and/or downstream of scrubber 104 (or of liquid generation assembly 106). For example, heater 230 or another heater configured to heat the ionic liquid mixture may not be present between an outlet of scrubber 104 and an inlet of stripper 108. In some examples, no additional heat in an amount sufficient to increase the temperature of the ionic liquid mixture is provided other than by vacuum pump 110. Thus, the ionic liquid mixture may be heated solely by absorbing heat from vacuum pump 110.

In the example of FIG. 4, contaminant removal system 200 may include one or more systems or components configured to further process contaminant stream 240. In some examples, contaminant removal system 200 includes a compressor (not shown), condenser 244, and water separator 246 configured to compress and/or condense contaminant stream 240 and remove water from the compressed or condensed contaminant stream 240. For example, for carbon dioxide removed from contaminant removal system 200 to be stored or recycled, the compressor, condenser 244, and water separator 246 may compress contaminant stream 240 to a high pressure and remove nearly all water from contaminant stream 240. In a life support application, a large amount of water may be present in cabin air stream 210. For example, the concentration of water in cabin air stream 210 may be much higher than that of carbon dioxide. Sabatier system 114 may require a water concentration of less than 2% to react hydrogen gas with carbon dioxide.

Contaminant stream 240 may be compressed by a compressor. A variety of compressors may be used including, but not limited to, centrifugal compressors, positive displacement compressors, and the like. Condenser 244 may be configured to cool contaminant stream 240 (e.g., after compression) and condense water from contaminant stream 240. For example, condenser 244 may be coupled to a refrigeration system or other cooling system that circulates a cooling medium to cool contaminant stream 240. A variety of condensers may be used for condenser 244 including, but not limited to, shell and tube heat exchangers, plate-fin, surface coolers, heat pipes, thermoelectric devices, cooling jackets, and the like. Water separator 246 may be configured to remove water from contaminant stream 240, discharge a dehumidified contaminant stream 248 to Sabatier system 114, and discharge contaminant water stream 252 to water storage 116. A variety of water separators may be used including, but not limited to, static phase separators, capillary phase separator, membrane phase separators, centrifugal/rotary separators, and the like.

Controller 118 may be communicatively coupled to and configured to receive measurement signals from the one or more sensor sets, and other process control components (not shown) of contaminant removal system 200, such as: vacuum pump 110; control valves for cabin air stream 210, clean air stream 216, supply air stream 218, contaminant stream 240, and inlets/outlets to heat exchanger 228, heater 230, ionic liquid storage 232, and cooler 236; pump 234; pump 238; blower 222; and the like.

The following enumerated clauses describe examples according to the present disclosure.

Clause 1: A contaminant removal system, including: a scrubber configured to absorb a contaminant from an air stream into an ionic liquid sorbent in an ionic liquid mixture; an ionic liquid regeneration assembly configured to desorb the contaminant from the ionic liquid sorbent under vacuum; and a vacuum pump configured to generate the vacuum in the ionic liquid regeneration assembly, where the vacuum pump is configured to transfer pump heat to the ionic liquid mixture.

Clause 2: The contaminant removal system of clause 1, where the vacuum pump defines a liquid inlet, a liquid outlet, and a liquid passage between the liquid inlet and the liquid outlet, and where the vacuum pump is configured to transfer the pump heat to the ionic liquid mixture along the liquid passage.

Clause 3: The contaminant removal system of clause 2, where the liquid passage is isolated from vacuum of the vacuum pump.

Clause 4: The contaminant removal system of clauses 2 or 3, where the vacuum pump includes a liquid conduit defining the liquid passage.

Clause 5: The contaminant removal system of clause 4, where the liquid conduit includes at least one metal or alloy.

Clause 6: The contaminant removal system of clause 5, where the liquid conduit includes one or more tubes.

Clause 7: The contaminant removal system of any of clauses 2 to 6, where the liquid inlet of the vacuum pump is fluidically coupled to the scrubber to receive the ionic liquid mixture from the scrubber at a first temperature, and where the liquid outlet of the vacuum pump is configured to discharge the ionic liquid mixture at a second temperature greater than the first temperature.

Clause 8: The contaminant removal system of clause 7, where the ionic liquid regeneration assembly includes a stripper configured to desorb the contaminant from the ionic liquid sorbent, and where the stripper is fluidically coupled to the liquid outlet of the vacuum pump to receive the ionic liquid mixture at a temperature greater than the first temperature.

Clause 9: The contaminant removal system of any of clauses 1 to 8, where the vacuum pump is configured to generate the pump heat when generating the vacuum.

Clause 10: The contaminant removal system of any of clauses 1 to 9, where the contaminant removal system does not include a heater configured to heat the ionic liquid mixture downstream of the scrubber and upstream of the ionic liquid regeneration assembly.

Clause 11: The contaminant removal system of clause 1, where the contaminant is carbon dioxide.

Clause 12: The contaminant removal system of clause 11, further including a Sabatier system coupled to the ionic liquid regeneration assembly and configured to generate hydrocarbons using the contaminant.

Clause 13: A method for removing a contaminant from an environment, the method including: absorbing, by a scrubber, a contaminant from an air stream into an ionic liquid sorbent in an ionic liquid mixture; applying, by a vacuum pump, vacuum in an ionic liquid regeneration assembly; desorbing, by the ionic liquid regeneration assembly under the vacuum, the contaminant from the ionic liquid sorbent; and transferring pump heat from the vacuum pump to the ionic liquid mixture.

Clause 14: The method of clause 13, further including introducing the ionic liquid mixture from the scrubber at a first temperature into the vacuum pump, and allowing the ionic liquid mixture to heat to a second temperature greater than the first temperature by absorbing the pump heat.

Clause 15: The method of clause 14, where the ionic liquid regeneration assembly includes a stripper, and where the method further includes receiving, by the stripper, the ionic liquid mixture at a temperature greater than the first temperature from the vacuum pump.

Clause 16: The method of clause 15, where the ionic liquid mixture is not heated in a heater outside the vacuum pump downstream of the scrubber and upstream of the stripper.

Clause 17: The method of any of clauses 13 to 16, where the vacuum pump includes a liquid conduit defining a liquid passage, and where transferring the pump heat includes flowing the ionic liquid mixture along the liquid passage.

Clause 18: The method of any of clauses 13 to 17, where the contaminant is carbon dioxide.

Clause 19: The method of clause 18, further including generating, by a Sabatier system, hydrocarbons using the desorbed contaminant.

Clause 20: A contaminant removal system, comprising: a scrubber configured to absorb a contaminant from an air stream into an ionic liquid sorbent in an ionic liquid mixture; a stripper configured to desorb the contaminant from the ionic liquid sorbent under vacuum; and a vacuum pump configured to generate the vacuum in the stripper, the vacuum pump defining a liquid inlet, a liquid outlet, and a liquid passage between the liquid inlet and the liquid outlet, wherein the scrubber is fluidically coupled to the liquid inlet of the vacuum pump, wherein the stripper is fluidically coupled to the liquid outlet of the vacuum pump, wherein the vacuum pump is configured to transfer pump heat to the ionic liquid mixture along the liquid passage, and wherein the liquid passage is isolated from vacuum of the vacuum pump.

Clause 21: A method for controlling a contaminant removal system, the method including: controlling, by control circuitry, a flow of an ionic liquid mixture including an ionic liquid sorbent through an ionic liquid circuit, where the ionic liquid circuit includes: a scrubber configured to absorb a contaminant from an air stream into the ionic liquid sorbent in the ionic liquid mixture; and an ionic liquid regeneration assembly configured to desorb the contaminant from the ionic liquid sorbent under vacuum; controlling, by the control circuitry, a vacuum pump to apply the vacuum to desorb the contaminant from the ionic liquid sorbent; and controlling, by the control circuitry, the flow of the ionic liquid mixture through the vacuum pump to transfer pump heat to the ionic liquid mixture.

The techniques described in this disclosure may be implemented, at least in part, in hardware, software, firmware, or any combination thereof. For example, various aspects of the described techniques, such as functionality attributed to controller 118 or the various sensors above, may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various techniques described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware, firmware, or software components. Rather, functionality associated with one or more modules or units may be performed by separate hardware, firmware, or software components, or integrated within common or separate hardware, firmware, or software components.

The techniques described in this disclosure may also be embodied or encoded in an article of manufacture including a computer-readable storage medium encoded with instructions. Instructions embedded or encoded in an article of manufacture including a computer-readable storage medium, may cause one or more programmable processors, or other processors, to implement one or more of the techniques described herein, such as when instructions included or encoded in the computer-readable storage medium are executed by the one or more processors. Computer readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may include one or more computer-readable storage media.

In some examples, a computer-readable storage medium may include a non-transitory medium. The term “non-transitory” may indicate that the storage medium is not embodied in a carrier wave or a propagated signal. In certain examples, a non-transitory storage medium may store data that can, over time, change (e.g., in RAM or cache).

Various examples have been described. These and other examples are within the scope of the following claims.

What is claimed is:

1. A contaminant removal system, comprising:
a scrubber configured to absorb a contaminant from an air stream into an ionic liquid sorbent in an ionic liquid mixture;
an ionic liquid regeneration assembly configured to desorb the contaminant from the ionic liquid sorbent under vacuum; and
a vacuum pump configured to generate the vacuum in the ionic liquid regeneration assembly,
wherein the vacuum pump is configured to transfer pump heat to the ionic liquid mixture.

2. The contaminant removal system of claim 1, wherein the vacuum pump defines a liquid inlet, a liquid outlet, and a liquid passage between the liquid inlet and the liquid outlet, and wherein the vacuum pump is configured to transfer the pump heat to the ionic liquid mixture along the liquid passage.

3. The contaminant removal system of claim 2, wherein the liquid passage is isolated from vacuum of the vacuum pump.

4. The contaminant removal system of claim 2, wherein the vacuum pump comprises a liquid conduit defining the liquid passage.

5. The contaminant removal system of claim 4, wherein the liquid conduit comprises at least one metal or alloy.

6. The contaminant removal system of claim 5, wherein the liquid conduit comprises one or more tubes.

7. The contaminant removal system of claim 2, wherein the liquid inlet of the vacuum pump is fluidically coupled to the scrubber to receive the ionic liquid mixture from the scrubber at a first temperature, and wherein the liquid outlet of the vacuum pump is configured to discharge the ionic liquid mixture at a second temperature greater than the first temperature.

8. The contaminant removal system of claim 7, wherein the ionic liquid regeneration assembly comprises a stripper configured to desorb the contaminant from the ionic liquid sorbent, and wherein the stripper is fluidically coupled to the liquid outlet of the vacuum pump to receive the ionic liquid mixture at a temperature greater than the first temperature.

9. The contaminant removal system of claim 1, wherein the vacuum pump is configured to generate the pump heat when generating the vacuum.

10. The contaminant removal system of claim 1, wherein the contaminant removal system does not comprise a heater configured to heat the ionic liquid mixture downstream of the scrubber and upstream of the ionic liquid regeneration assembly.

11. The contaminant removal system of claim 1, wherein the contaminant is carbon dioxide.

12. The contaminant removal system of claim 11, further comprising a Sabatier system coupled to the ionic liquid regeneration assembly and configured to generate hydrocarbons using the contaminant.

13. A method for removing a contaminant from an environment, the method comprising:
absorbing, by a scrubber, a contaminant from an air stream into an ionic liquid sorbent in an ionic liquid mixture;
applying, by a vacuum pump, vacuum in an ionic liquid regeneration assembly;
desorbing, by the ionic liquid regeneration assembly under the vacuum, the contaminant from the ionic liquid sorbent; and
transferring pump heat from the vacuum pump to the ionic liquid mixture.

14. The method of claim 13, further comprising introducing the ionic liquid mixture from the scrubber at a first temperature into the vacuum pump, and allowing the ionic liquid mixture to heat to a second temperature greater than the first temperature by absorbing the pump heat.

15. The method of claim 14, wherein the ionic liquid regeneration assembly comprises a stripper, and
wherein the method further comprises receiving, by the stripper, the ionic liquid mixture at a temperature greater than the first temperature from the vacuum pump.

16. The method of claim 15, wherein the ionic liquid mixture is not heated in a heater outside the vacuum pump downstream of the scrubber and upstream of the stripper.

17. The method of claim 13, wherein the vacuum pump comprises a liquid conduit defining a liquid passage, and
wherein transferring the pump heat comprises flowing the ionic liquid mixture along the liquid passage.

18. The method of claim 13, wherein the contaminant is carbon dioxide.

19. The method of claim 18, further comprising generating, by a Sabatier system, hydrocarbons using the desorbed contaminant.

20. A contaminant removal system, comprising:
a scrubber configured to absorb a contaminant from an air stream into an ionic liquid sorbent in an ionic liquid mixture;
a stripper configured to desorb the contaminant from the ionic liquid sorbent under vacuum; and
a vacuum pump configured to generate the vacuum in the stripper, the vacuum pump defining a liquid inlet, a liquid outlet, and a liquid passage between the liquid inlet and the liquid outlet,
wherein the scrubber is fluidically coupled to the liquid inlet of the vacuum pump,
wherein the stripper is fluidically coupled to the liquid outlet of the vacuum pump,
wherein the vacuum pump is configured to transfer pump heat to the ionic liquid mixture along the liquid passage, and
wherein the liquid passage is isolated from vacuum of the vacuum pump.
